(19) United States
(12) Patent Application Publication
(10) Pub. No.: US 2022/0008326 A1
(43) Pub. Date: Jan. 13, 2022

(54) RA Facial Moisturizer

(71) Applicant: Tova Palomino, Coral Springs, FL (US)

(72) Inventor: Tova Palomino, Coral Springs, FL (US)

(21) Appl. No.: 16/922,285

(22) Filed: Jul. 7, 2020

(51) Int. Cl. A61K 8/9789; A61K 8/9794; A61Q 19/08

(52) U.S. Cl. CPC A61K 8/9789; A61K 2800/59; A61Q 19/08; A61K 8/9794

(57) ABSTRACT

The present invention discloses an organic skin moisturizer, which extends and improves the skin cells. The composition is dermatologically acceptable and is capable of moisturizing skin for at least twelve hours after topical application to skin even after multiple washings of the skin.

### BACKGROUND

### Copyright Notice

A portion of the disclosure of this patent document contains material which is subject to copyright protection The copyright owner has no objection to the facsimile reproduction by anyone of the patent document or the patent disclosure, as it appears in the Patent and Trademark Office patent file or records, bit otherwise reserves all copyright rights whatsoever.

### FIELD OF THE INVENTION

The present invention pertains generally relates to cosmetic preparations for the moisturization of human skin. More particularly, the present invention is particularly, but not exclusively, useful for a moisturizing skin cream suitable for use on the face and neck.

### DESCRIPTION OF THE RELATED ART

The following background information may present examples of specific aspects of the prior art (e. g., without limitation, approaches, facts, or common wisdom) that, while expected to be helpful to further educate the reader as to additional aspects of the prior art, is not to be construed as limiting the present invention, or any embodiments thereof to anything stated or implied therein or inferred thereupon.

There are many products and formulas for treating dry rough skin and just as many formulas to fight wrinkles. The prior art is replete with products and patents claiming skin protection and salvations for aging skin however the present invention combines a combination of organic ingredients for treating various skin disorders that is unique and effective in bringing about soft, moist skin and reducing wrinkles without harming the skin or the body.

Skin disorders, as the term is used herein, encompasses numerous skin conditions ranging in severity from severe dermatitis, severe dry skin, psoriasis, rosacea and other skin blemishes to less severe conditions, such as lack of adequate skin firmness, dermal hydration or scaling, which are nonetheless unsightly and may cause physical discomfort.

In addition, it is known that proper skin nutrition, characterized by the adequate supply and consistent replenishment of certain vitamins to the skin can both reduce the effects that environmental factors have on the skin, and may also reverse the signs of premature skin aging. Further, it is known that improper skin nutrition due to a deficiency of certain vitamins can cause premature aging, even in the absence of other environmental factors.

There are numerous patents issued for the care and condition of skin Each patent uses a variety of ingredients with the combination providing some form of skin treatment. The list is exhaustive, and includes such patents as: U.S. Pat. No. 7,306,810 issued Dec. 11, 2007 to Spencer discloses a skin cream which comprises at least one anti-oxidant, an anti-inflammatory agent, an exfoliant, and an agent to protect against UV irradiation and the cream is made from either oils or creams to promote adsorption of the active ingredients and to increase vibrancy of the skin tone. U.S. Pat. No. 6,281,236 issued Aug. 28, 2001 to Farber discloses a skin cream composition containing allantoin and an emulsifier with improved stability coming from the adjustment of the pH to a range of 4.5 to 5.8. The lower pH preserves the stability of the allantoin and the functionality of the emulsifier system is maintained. 6,572,868, issued Jun. 3, 2003 to Sandra E. Cope, discloses a restructuring complex for cosmetic compositions. The composition comprises safe and effective amounts of carrageenans, borage seed oil, squalane, ceramide 3, ceramide 6, red algae extract, dipalmitoyl hydroxyproline, and oleuropein. U.S. Pat. No. 6,193,987, issued Feb. 27, 2001 to M. H. Harbeck, discloses a lubricating composition for the hands and skin. The composition has as its constituents a mixture of organic safflower oil, flaxseed oil, tincture of benzoin, and organic beeswax. And, U.S. Pat. No. 6,479,043, issued Nov. 12, 2002 to Tietjen et al., discloses a depilatory composition. The composition includes emollients, skin conditioners, buffering agents, viscosity increasing agents, emulsion stabilizers, pH adjusters, chelating agents, fragrance, color, lubricants, propellants, or biological agents.

Various topical formulations and oral regimens of vitamins and herbs have been proposed for the treatment of skin conditions. U.S. Pat. No. 6,228,387, issued May 8, 2001 to M. Borod, describes a first composition for topical application and a second composition for oral administration for the treatment of hemorrhoids. The topical composition includes several herbs and vitamins, including grape seed extract and vitamin E, and in one embodiment, a few drops of Essential Oil of Chamomile. Vitamin E occurs naturally as a mixture of tocopherols, the most active being α-tocopherol, used externally, vitamin E is healing to the skin, being used for protection from sun damage, reducing facial lines and wrinkles, and improving skin smoothness, being used as an additive to massage oils and face creams.

In light of the above it is an object of the present invention to provide a safe and effective skin cream suitable for the purposes of softening the skin and preventing the premature aging of skin. It is another object of the present invention to provide a moisturizing skin cream that effectively delivers vitamins to the skin to ensure proper skin nutrition. It is yet another object of the present invention to provide a moisturizing skin cream suitable for application to the face and neck area. Yet another object of the present invention is to provide a moisturizing skin cream which is easy to use, relatively simple to manufacture, and comparatively cost effective.

The present invention provides a new moisturizer form having many of the properties of an ideal moisturizer. For example, the lotion provides excellent moisturization and increases the cell turnover rate on the stratum corneum without irritation. None of the prior art methods have been found to be completely suitable to meet these needs and are cumbersome. The present invention provides such a method and the overall combination of these features is nowhere disclosed in the prior art cited above which appears to be representative of the general art in this area although it is not intended to be an all-inclusive listing of pertinent prior art patents.

### SUMMARY

In fight of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

The present invention is a facial moisturizer which is to be applied to the face and neck. The formula is a mixture of oils and essential oils which is to help moisturize and aid in common skin concerns.

Another embodiment of the invention is a mixture of oils and essential oils which is to help moisturize and aid in common skin problems.

Another object of the present invention is to provide a cosmetic preparation such as moisturizer which produce products that have long lasting effects on the skin and results in longer effective treating times for any active ingredient which may be added.

In an alternative embodiment, the present invention effectively softens, moisturizes and provides a unique essential oils combination of needed nutrients to the skin.

Advantage of this invention. The presence of these multiple active natural ingredients together provide a very rich nourishing, moisturizing and anti-aging cream giving a faster and long lasting effect on the skin.

It is principal object of the invention, by effectively softening, moisturizing, and supplying appropriate vitamins to the skin, the skin cream of the present invention effectively helps to prevent the premature aging of skin.

Another significant embodiment of the present invention provide an advantageously a single composition which moisturizes the skin and protect it at the same time. Thus, it is more convenient for a subject in need of such a composition and advantageously provide a great compliance. One embodiment of the present invention is a moisturizing composition comprising at least one moisturizer ingredient.

It is an object of the invention to provide a new skin moisturizer which may be easily and efficiently manufactured and marketed and which is of an effective and reliable construction.

The essential oils used herein according to present invention is selected from

### Vitis Vinifera Oil (Grapeseed)

### Rosa Moschata Seed Oil (Rosehip)

### Daucus Carota Stavia Seed Oil (Carrot)

### Prunus Dulcis Oil (Sweet Almond)

### Boswellia Carterii Oil (Frankincense)

### Lavandula Agustifolia Oil (Lavender)

### Pelargonium Graveolens Oil (Geranium)

### Cymbopogon Fleuosus Oil (Lemongrass)

### Helichrysum Italicum Oil (Immortelle)

It is an object of the invention to provide a new skin moisturizer which provides the following benefits:

### Deep Moisturizer

Astringent (kills harmful germs and bacteria)
Balances hormones levels
Non-comedogenic (Less likely to clog pores and cause breakouts)
Tighten pores
Prevents acne and blackheads
Reduces dark circles, dark spots, age spots, scars, fine lines and wrinkles
SPF protection

### Anti-aging

### Anti-fungal

Rejuvenates skin
Restores collagen

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description It is also to be understood that the specific devices and processes described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

Unless otherwise defined, all terms (including technical and scientific terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques are disclosed. Each of these has individual benefit and each can also be used in conjunction with one or more, or in some cases all, of the other disclosed techniques. According, for the sake of clarity, this description will refrain from repeating possible combination in an unnecessary fashion. Nevertheless, the specifications and claims should be read with the understanding that such combinations are entirely within the scope of the invention and the claim/s.

The method of the present invention is that it relates to a facial moisturizer which is to be applied to the face and neck. The formula is a mixture of oils and essential oils which is to help moisturize and aid in common skin concerns. The particular formula consists of the following:

### Vitis Vinifera Oil (Grapeseed)

### Rosa Moschata Seed Oil (Rosehip)

### Daucus Carota Stavia Seed Oil (Carrot)

### Prunus Dulcis Oil (Sweet Almond)

### Boswellia Carterii Oil (Frankincense)

### Lavandula Agustifolia Oil (Lavender)

### Pelargonium Graveolens Oil (Geranium)

### Cymbopogon Fleuosus Oil (Lemongrass)

### Helichrysum Italicum Oil (Immortelle)

It is an object of the invention to provide a new skin moisturizer which provides the following benefits:

### Deep Moisturizer

Astringent (kills harmful germs and bacteria)
Balances hormones levels
Non-comedogenic (Less likely to clog pores and cause breakouts)
Tighten pores
Prevents acne and blackheads
Reduces dark circles, dark spots, age spots, scars, fine lines and wrinkles
SPF protection

### Anti-aging

### Anti-fungal

Rejuvenates skin
Restoes collagen.

Another embodiment of the present invention relates to simple application where the moisturizing skin cream can be applied daily. To apply, a generous amount is first placed on the fingertips or an appropriate delivery device such as a sponge or cloth. Next, the cream is dabbed onto the skin at the area to be treated. Finally, the cream is rubbed into the skin using the fingertips until the cream becomes transparent Use of the skin cream immediately after washing and towel drying the skin is recommended for maximum moisturizing effect

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Moreover, a latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

1) The invention is a formula of the facial moisturizer with essential oils comprising:
a. Vitis Vinifera Oil (Grapeseed);
b. Rosa Moschata Seed Oil (Rosehip);
c. Daucus Carota Stavia Seed Oil (Carrot);
d. Prunus Dulcis Oil (Sweet Almond);
e. Boswellia Carterii Oil (Frankincense);
Lavandula Agustifolia Oil (Lavender);
g Pelargonium Graveolens Oil (Geranium);
h. Cymbopogon Fleuosus Oil (Lemongrass);
i Helichrysum Italicum Oil (Immortelle).

2) wherein the invention is Nourishing face moisturising lotion, Revitalizing hand and body moisturizer providing following benefits:
a. Deep Moisturizer;
b. Astringent (kills harmful germs and bacteria);
c. Balances hormones levels;
d. Non-comedogenic (Less likely to clog pores and cause breakouts);
e. Tighten pores;
f. Prevents acne and blackheads;
g. Reduces dark circles, dark spots, age spots, scars, fine lines and wrinkles;
h. SPF protection;
i Anti-aging;
j. Anti-fungal;
k. Rejuvenates skin;
l. Restores collagen.

3) The invention can be used multiple times a day and preferably for use in morning and night.
